(19) United States
(12) Patent Application Publication
(10) Pub. No.: US 2017/0027447 A1
(43) Pub. Date: Feb. 2, 2017

(54) SYSTEM AND METHOD FOR IMPROVED LIGHT DELIVERY TO AND FROM SUBJECTS

(71) Applicant: THE GENERAL HOSPITAL CORPORATION, Boston, MA (US)

(72) Inventors: F Jason Sutin, Cambridge, MA (US); Pei-Yi Lin, Cambridge, MA (US); Maria A. Franceschini, Winchester, MA (US)

(21) Appl. No.: 15/303,456

(22) PCT Filed: Apr. 17, 2015
(86) PCT No.: PCT/US15/26455

§ 371 Date: Oct. 11, 2016

Related U.S. Application Data
(60) Provisional application No. 61/981,300, filed on Apr. 18, 2014.

(51) Int. Cl. A61B 5/00; G01J 3/02; F21V 8/00; G02B 27/09; G02B 5/04; G01J 3/10; G02B 5/02

(52) U.S. Cl. CPC A61B 5/0075; G01J 3/108; G01J 3/0218; G02B 5/0278; G02B 27/09; G02B 5/04; G02B 6/001; A61B 2562/0233

(57) ABSTRACT

An optical probe comprising a light source providing a light that is directed along a first axis; a diffusive element positioned proximate to the light source to receive the light and to diffuse the light as it exits the diffusive element; and a directional optical element directing the light exiting the diffusive element along at least one of the first axis and a second axis generally perpendicular to the first axis to project the light out of the optical probe and onto a subject.

### CROSS-REFERENCE TO RELATED APPLICATIONS

This application is based on, claims priority to, and incorporates herein by reference, United States provisional patent Application Serial No. 61/981,300 filed Apr. 18, 2014.

### STATEMENT REGARDING FEDERALLY SPONSORED RESEARCH

Not Applicable

### BACKGROUND

Modern medical diagnostic equipment allows for non-invasive ways for gathering and analyzing human biological data. For example, data such as blood and/or tissue oxygenation levels, blood sugar levels, intracranial bleeding scans, monitoring anesthesia and surgical procedures, and the like, can be performed using non-invasive medical diagnostics. A widely-used method of obtaining medical data using non-invasive techniques, involves using spectroscopy, and particularly, near-infrared spectroscopy (NIRS). Various types of NIRS can be used to obtain spectroscopic measurements. For example, types of NIRS systems can include continuous wave (CW), time-resolved (TR), frequency domain (FD), time domain (TD) NIRS and diffuse correlation spectroscopy (DCS).

NIRS systems require a light, generally in the near-infrared spectrum, to be delivered to a patient from a light source. The light source can be remote from the patient or in close proximity. Further, the light source can use intermediate optics to tailor the light to the specific application. In a standard implementation, a focused laser or a fiber optic element can be directly applied to a patients skin. However, this direct interaction between the light source and the patient can have some safety and performance disadvantages.

For example, exposure to certain light types (e.g. infrared, near-infrared, and the like), at certain power levels, can create a safety issue for patients. Light exposure safety is generally recognized to depend on the magnitude of the light power and the amount of surface area illuminated by the light source. For example, the American National Standards Institute (ANSI) provides a guideline for the safe use of lasers, which is a widely used standard for light exposure safety determinations. Specifically, the ANSI standard determines safe light exposure levels based on a maximum amount of optical power (Watts) exposure allowed per square centimeter of human tissue (i.e. power density). This provides clear guidance for determining safe illumination levels for NIRS diagnostic tools. Furthermore, ANSI also provides standards relating to eye safety and light power. Specifically, ANSI standards require a minimum amount of angular divergence of the light transmitted by a light source such that the human eye cannot focus the light source above a maximum permissible power (Watts) per square centimeter of retina. While ANSI standards are not required to be followed in certain applications, similar concepts apply as excessive light power density can cause burns, combustion, ablation, and/or other adverse effects on a patient.

Currently, NIRS systems typically use light sources and/or fiber optics with very small cross-sectional areas, resulting in high light power density. Additionally, the angular divergence of these small cross sectional areas light source is typically small. Accordingly, where these light sources are used directly, the total light power must be kept low to ensure patient safety. However, this can often result in low signal-to-noise ratios, which can lead to a degradation of the accuracy of the diagnostic information.

### SUMMARY

The present disclosure provides systems and methods for increasing light throughput through an optical probe while maintaining safe exposure levels for a subject.

Specifically, and in accordance with one aspect of the present invention, an optical probe is provided. The optical probe comprises a light source providing a light that is directed along a first axis. The optical probe further comprises a diffusive element positioned proximate to the light source to receive the light and to diffuse the light as it exists the diffusive element; and a directional optical element directing the light exiting the diffusive element along the first axis or a second axis generally perpendicular to the first axis to project the light out of the optical probe and onto a subject.

In accordance with another aspect of the present invention, a method of increasing light throughput in an optical probe is provided. The method comprises transmitting alight along a first axis from a light source; and receiving the light through a diffusive element, the diffusive element positioned proximate to the light source to diffuse the light as it exits the diffusive element. The method further comprises directing the light using a directional element, the directional element directing the light exiting the diffusive element along at least one of the first axis and a second axis generally perpendicular to the first axis, to project the light out of the optical probe and onto a subject.

In accordance with yet another aspect of the present invention, a side lit optical spectroscopy device is presented. The device comprises a light source providing a light that is directed along a first axis into a light guide. The device further comprises a reflective element, the reflective element positioned proximately along a first side of the light guide and configured to reflect the light from the light source towards a second side of the light guide; and a scattering layer, the scattering layer positioned proximate to the second side of the light guide and configured to scatter the light from the light source and the light reflected by the reflective element prior to the light exiting the side lit optical spectroscopy device.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The invention will be better understood and features, aspects and advantages other than those set forth above will become apparent when consideration is given to the following detailed description thereof. Such detailed description makes reference to the following drawings.

FIG. 1 is a system view of a prior art optical probe.

FIG. 2 is a system view of an optical probe with a diffusive element.

FIG. 3 is a light transmission chart illustrating diffusion of light using various methods of diffusion.

FIG. 4 is a system diagram illustrating a scrambling device.

FIG. 5 is a series of data plots showing optical output using different launching techniques.

FIG. 6 is a data plot showing the effects of different transmission modes on step-index multimode fiber optic cables.

FIG. 7 is a system view of a side-lit optical probe.

FIG. 8 is a system view of a side-lit optical probe having a scattering layer.

FIG. 9 is a system view of a side-lit optical probe with a light source embedded in the light guide.

FIG. 10 is a system view of a side-lit optical probe with a light source embedded in the light guide and a scattering layer.

FIG. 11 is a system view of a side-lit optical probe with an angular light guide.

FIG. 12 is a system view of a side-lit optical probe with an angular light guide and a scattering layer.

FIG. 13 is a system view of a side-lit optical probe with a plurality of scattering devices.

FIG. 14 is a system view of a side-lit optical probe with a plurality of scattering devices and a scattering layer.

FIG. 15 is a system view of a side-lit optical probe with a light source embedded in the light guide and a plurality of scatting devices.

FIG. 16 is a system view of a side-lit optical probe with a light source embedded in the light guide, a plurality of scatting devices, and a scattering layer.

FIG. 17 is a system view of a side-lit optical probe with an angular light guide and a plurality of scattering devices.

FIG. 18 is a system view of a side-lit optical probe with an angular light guide, a plurality of scattering devices and a scattering layer.

### DETAILED DESCRIPTION

As discussed, optical spectroscopy, particularly NIRS, uses light to gather and determine certain biological data in patients. NIRS, while allowing for non-invasive diagnostic capability, must ensure that the magnitude of the optical illumination power, and, in some cases, the angular divergence, can remain below certain levels to avoid harm to a patient's tissue. Therefore, devices and methods are needed to increase the signal-to-noise ratio of NIRS devices by delivering as much light as possible to a subject without exceeding safety limits for light exposure. The below devices, systems and methods improve light delivery over existing methods to reduce the amount of power required from a light source, and can further increase the total amount of light power permitted to be applied to a subject.

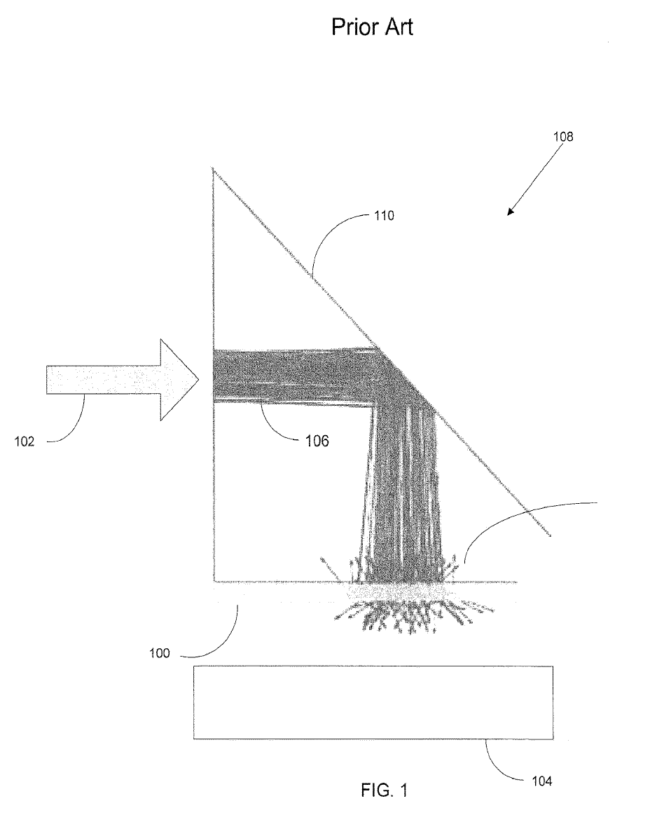

FIG. 1 shows a prior art system that includes a diffuser 100 between a light source 102 and a subject 104. The light source 102 can generate a light 106. The subject 104, in this example could be human tissue. Alternatively, the subject 104 can be other biological tissue. Further, the subject 104 can be free space. In one example, the diffuser 100 can be a sheet of Teflon. However, it should be known that other applicable diffuser elements could be used as applicable. The diffuser 100 is positioned over the source exit on a diagnostic device 108. Additionally, an optional intermediate optic 110 can be installed between the light source 102 and the diffuser 100. In one example, the optional intermediate optic 110 is a prism. The diffuser 100 increases the angular divergence of the light 106, thereby increasing eye safety. Where the diagnostic device 108 is in contact with the subject 104, the diffuser 100 can be between the light source 102 and the subject 104. The diffuser 104 can scatter the light 106 from the light source 102 within the volume of the diffuser 100 to increase the cross-sectional area of the light 106 when the light 106 passes through the diffuser 100, which is known in the art as volumetric scattering. This increase in cross-sectional area can allow for a higher power light source to be used without exceeding maximum light power density requirements. This, in turn can increase the signal-to-noise ratio of the diagnostic device 108.

While the above-described system can increase the cross-sectional area of the light 106, there are limitations associated with volumetric scattering. First, volumetric scattering can yield a large amount of undesirable backscattered light. Backscattered light can reduce the amount of forward scattered light delivered to the subject 104. Second, volumetric scattering relying on a diffuser 100 between the light source 102 and the subject 104 only generates a small increase in the cross-sectional area of the light output from the diagnostic device 108. Where the diffuser 100 is a Teflon sheet, the cross-sectional area can be increased by increasing the thickness of the Teflon sheet. However, increase in the thickness of a Teflon sheet can result in increased backscattered light, thereby reducing the power of light delivered to the subject 104. Additionally, diffusers 100 such as Teflon sheets located immediately adjacent to the subject 104, as illustrated in FIG. 1, are exposed and therefore can degrade. Degradation of the Teflon sheet can result in a decrease in the volumetric scattering effect, thereby allowing the diagnostic device 108 to output light 106 at a power density that exceeds safety limits. While the above description discusses a Teflon sheet used as the diffuser, other diffusing elements with similar properties can be used as well as the diffuser 100. Thus, a solution to allow for increased light source power while maintaining output light density at safe levels is needed.

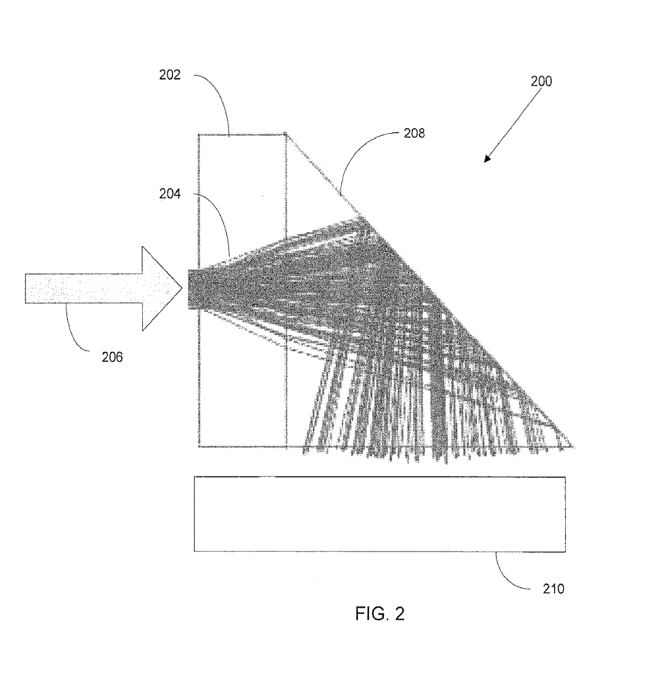

Turning now to FIG. 2, an optical probe 200 with a diffusive element 202 can be seen. In one configuration, the optical probe 200 can be a NIRS device. However, the optical probe 200 can be other types of spectroscopy devices or other photo-exciting and/or photo-illuminating devices. The diffusive element 202 can provide a controlled diffusion effect to expand and direct a light 204 from a light source 206. Non limiting examples of possible light sources 206 can include lasers, incandescent lamps, LEDs, fiber optic cables, light guides, or the like. Non-limiting examples of diffusive elements 202 can include surface, diffractive, refractive, holographic, surface holographic, and phase diffusive elements. While each of the non-limiting examples of diffusive elements can be used, they can be selected based on the type of application. For example, surface and/or surface holographic diffusive elements generally have a lower cost, but can be more complicated to use in an application having an integrated fiber optic design due to the general requirement of a free-space segment. Conversely, holographic or phase diffusers can be relatively easy to use in an integrated fiber optic design, but can have a higher cost. The diffusive element 202, in one configuration, can be a weakly diffractive element. In one configuration, the diffusive element 202 can be integrated into the optical probe 200. Alternatively, the diffusive element 202 can include discrete components that can be applied to the optical probe 200. Further, multiple diffusive element types (i.e. surface, diffractive, refractive, holographic, phase diffusive, and the like) can be combined for use with a single optical probe 200. The diffusive element 202 can homogenize and/or beam shape the light 204. The homogenization and/or beam shaping of the light 204 can result in flattop or spatial conversion of the light 204.

Additionally, the diffusive element 202 can be used alone, or in conjunction with other optical elements within the optical probe 200. For example, in one configuration a prism 208 can optionally be placed between the diffusive element 202 and a subject 210. In one example, the prism 208 can be used to change the direction of the light 204. Changing the direction of the light 204 can be used to reduce the size of an optical probe 200 where the transmission or reception of the light 204 is perpendicular to the subject 210. In one embodiment, the prism 208 can be used to fold the direction of the light by 90 degrees. However, the prism 208 can fold the direction of the light 204 by more than 90 degrees or less than 90 degrees. In one embodiment, the prism 208 can fold the light 204 by 0 degrees. Further, the prism 208 can fold the light 204 by 180 degrees. Folding the light 204 by a certain angle can be used where a greater spread of the light 204 on the subject 210 is desired. For example, where the subject 210 is a human head, the probe may be flexible to follow the contours of the head, such as by using flexible fiber optic cables to transmit the light 204. By including prisms 208, the light can be directed onto the subject 210 instead of following the contour of the fiber optic cables. Contouring to the shape of the subject 210, can increase the subject's 210 comfort while also allowing for increased adherence and reduced motion of the probe, thereby increasing the accuracy of the optical probe 200. Alternatively, other optical elements, such as prism 208, can be used to direct the light along the same axis as the light 204 is transmitted by the light source 206.

Alternatively, one or more intermediate optics can be placed between the diffusive element 202 and subject 210. For example, intermediate lenses can be used to transform, project, magnify, minify, etc. the light 204. The intermediate lenses can be used in conjunction with or in place of prism 208. Furthermore, intermediate devices such as filters, attenuators, etc., could also be used. Additionally, in some configurations the light 204 can pass directly from the diffusive element 202 to the subject 210. In some configurations, such as that shown in FIG. 2, the diffusive element 202 can be positioned on an outer surface of the optical probe 200. Alternatively, the diffusive element 202 can be positioned behind or between other optical elements (for example, a window element, not shown) to prevent damage or wear to the diffusive element 202.

As shown in FIG. 2, the diffusive element 202 may be placed between the light source 206 and the subject 210. In one configuration, the diffusive element 202 may be arranged proximate to the light source 206. To this end, the diffusive element 202 may be arranged such that no other components or structures are arranged between the light source 206 and the diffusive element 202. The diffusive element 202 can increase the angles of the light 204 received from the light source 206. Thus, light source 206 provides a light that is directed along a first axis toward diffusive element 202, which is arranged along the first axis and proximate to the light source 206. As such, the diffusive element 202 receives the light through a first planar surface formed generally perpendicular to the axis to diffuse the light as it exits the diffusive element 202.

Table 1, shown below, illustrates the advantages of using a diffusive element, such as that discussed above, positioned adjacent or proximate to the light source over using a diffuser adjacent to the subject, as shown in FIG. 1.

TABLE 1
Diffusive Element vs. Teflon Sheet Diffusers

| | | % | Insertion |
|---|---|---|---|
| | | Transmission | Loss |
| 125 micron | Measured | 53% | 2.8 db |
| Teflon Sheet | | | |
| | Literature | 42% | 3.8 db |
| | Value | | |
| | Optical | 47% | 3.2 db |
| | Model | | |
| 250 micron | Measured | 40% | 4.0 db |
| Teflon Sheet | | | |
| | Literature | 31% | 5.1 db |
| | Value | | |
| | Optical | 30% | 5.2 db |
| | Model | | |
| Diffusive | Measured | 92% | 0.4 db |
| Element | | | |
| | Literature | 92% | 0.4 db |
| | Value | | |
| | Optical | — | — |
| | Model | | |

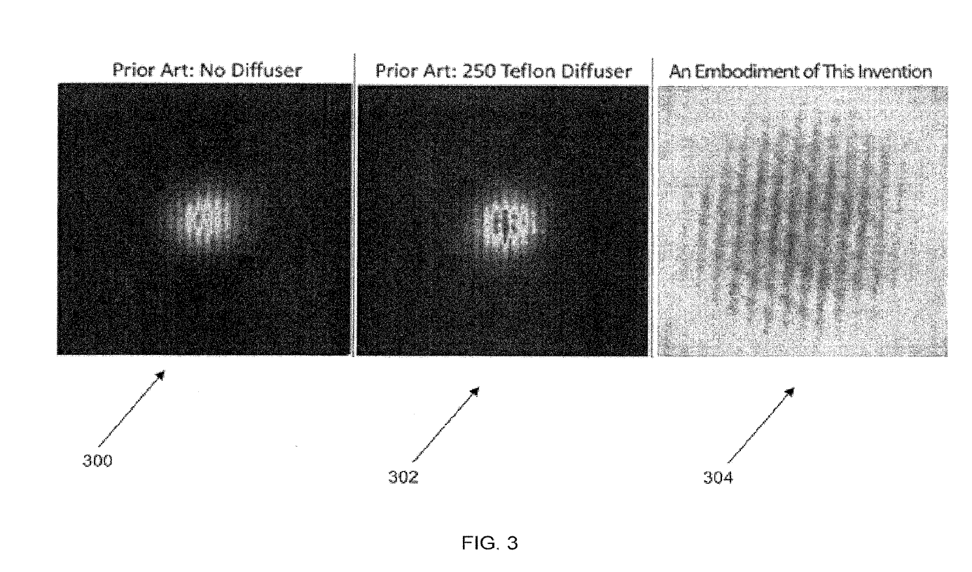

Looking at Table 1, it can be seen that the diffusive element positioned adjacent to the light source provided significantly higher percentages of light transmission and substantially reduced insertion loss over the prior art. Additionally, FIG. 3 illustrates three separate measured optical flux distributions. Result 300 was obtained using an optical probe having no diffuser, result 302 was obtained using an optical probe with a 250 micron Teflon diffuser, as used in the system of FIG. 1, and result 304 was obtained using a diffusive element, as shown above in FIG. 2. As can be seen, result 304 using the diffusive element provided substantially greater distribution of light distribution, resulting in a 6000% increase in maximum permissible exposure over the existing systems. This increase in maximum permissible exposure can result in as much as an 800% increase in signal-to-noise ration (SNR) of a NIRS device.

Referring again to FIG. 2, in some configurations, the optional prism 208 can be used to direct the light to the subject 210. The increase in the angles of the light 204 can substantially reduce the power density with minimal backscatter of the light 204 prior to it reaching the subject 210. In one example, the increase in the angle of the light 204 using a holographic diffusive element 202 can increase the optical transmission to the subject 210 by about 300% above the optical transmission possible using volumetric scattering techniques. Furthermore, the increase in spread of the light 204 over that achieved using volumetric scattering techniques can be about a 600% increase.

Continuing with FIG. 2, the optical probe 200 is shown with only a single light source. However, in some configurations multiple light sources 206 can be used. Furthermore, in one example, each of the multiple light sources 206 can transmit light 204 at the same wavelength. Alternatively, the multiple light sources 206 can transmit light at multiple wavelengths. The multiple light sources 206 can also transmit light that is pulsed or modulated. Safety standards, such as those promulgated by ANSI, set permissible exposure based on the total power density delivered by all sources. Therefore, if the multiple light sources 206 overlap on the subject 210, the power for each light source 206 can be additive in the overlapping regions. In some configurations, it is desirable to overlap the light sources 206 such that the light from the multiple light sources 206 probes the same area of the subject 210. By allowing for overlap from multiple light sources 206, optical probes can be miniaturized.

In another configuration, the light output of the optical probe 200 can use forms of light conversion to increase the total permissible optical power exposure output by the optical probe 200. As stated above, safety regulations provide a permissible threshold based on optical power density. Thus, one possible way to increase the total optical power output is to spread the delivered power over a greater area, thus reducing the power density. However, larger areas of illumination can be undesirable for NIRS based measurements. By using light conversion methods, a greater permissible power output can achieved by uniformly coving the subject area without peaks or “hotspots.”

To achieve conversion and/or homogenization of a light source, the light sources 206 can be launched into fiber optic cables or light guides where the light source 206 does not illuminate all modes of the fiber optic cable or light guide. Where the light source 206 does not illuminate all modes of the fiber optic cable or light guide, the profile of the light source 206 can be impressed on the distributions of modes in the fiber optic cable or light guide excited by the light source 206. In one example, where the light source 206 does not illuminate all modes of the fiber optic cable or light guide both the spatial size and angular spread of the light delivered by the fiber optic cable or light guide can be limited by the light source 206, and not the fiber optic cable or light guide. This can result in the power density being greater and non-uniform with one or more hotspots, thereby reducing the total permissible optical exposure.

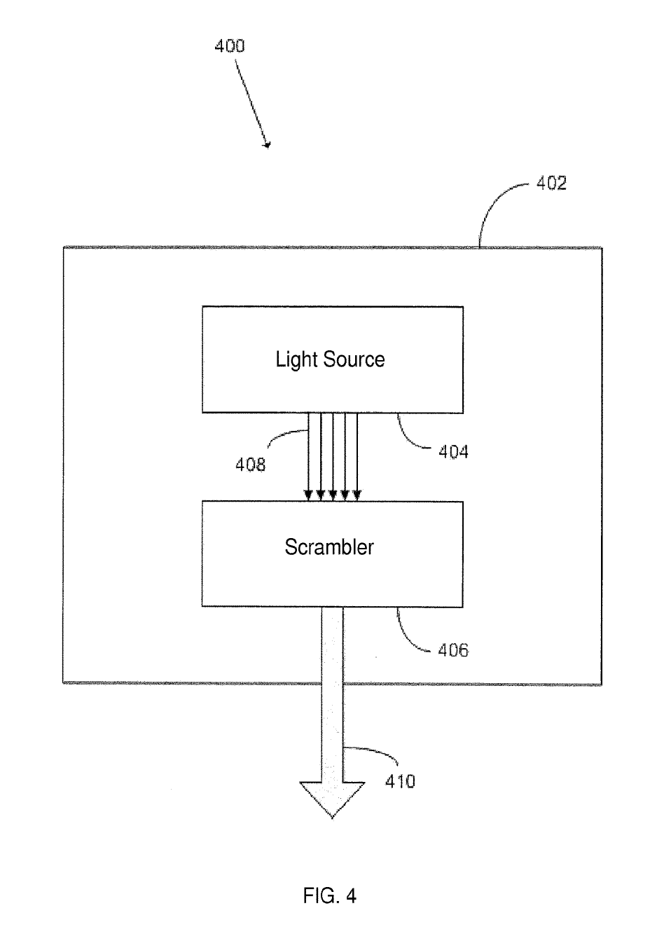

In one configuration, conversion methods can be used to transform light 204 guided using fiber optic cables or light guides as discussed above. Light 204 that is guided using fiber optic cables or light guides can be orthogonal, or nearly orthogonal. This can cause the light emitted to not interconvert, or to do so very slowly. This can cause the light leaving a fiber optic cable to be similar to the mode of the light source 206 and not the modes of the fiber optic cable or light guide. In one example, a fiber mode scrambler can be used to convert the light 204. An example fiber mode scrambling device 400 can be seen in FIG. 4. In one configuration, fiber mode scrambling device 400 can be integrated within optical probe 402. In one configuration, optical probe 402 can be a NIRS device. Additionally, the optical probe 402 can contain a light source 404 and a scrambler 406. The scrambler 406 can act on fiber optic cables to break the orthogonality of the fiber modes, allowing light to rapidly interconvert between multiple fiber modes. The scrambler 406 can, in some embodiments, expand the light to fill all of the propagating modes available in the fiber optic cable. Further, the scrambler 406 can reduce or eliminate the light from filling the non-propagating modes.

The scrambler 406 can receive a light 408 from the light source 404. Once the scrambler 406 has received the light 408, the scrambler 406 can perform a scrambling operation on the light 408, and output scrambled light 410. In one embodiment, the light source 404 can launch light into a light guide, such as a fiber optic cable, which can then be input into the scrambler 406 Alternatively, the light source 404 can launch light into separate fiber optic cable segments. The scrambler 406 can then perform a scrambling operation on the light in the fiber optic cable, which can result in a more equal distribution of light throughout the fiber optic cable. In one configuration, the light can be output using a fiber optic cable. Alternatively, the light can be output as a laser beam through free space. Where the scrambled light is output via a fiber optic cable, the output scrambled light 410 can fill a greater number of modes of the fiber optic cable. Further, the scrambled light 410 can expand more uniformly across the core of the fiber optic cable. This can result in a greater spatial and angular uniformity in the light output. This increased spatial and angular uniformity can improve light delivery to a subject.

In one embodiment, the scrambler 406 can apply a force on a fiber optic cable to bend and elastically distort the fiber optic cable such that the modes can become highly coupled. Similarly, the scrambler 406 can be used with waveguides, light guides, etc. Examples of these scramblers 406 can include microbending, corrugated, and single-point loading scramblers.

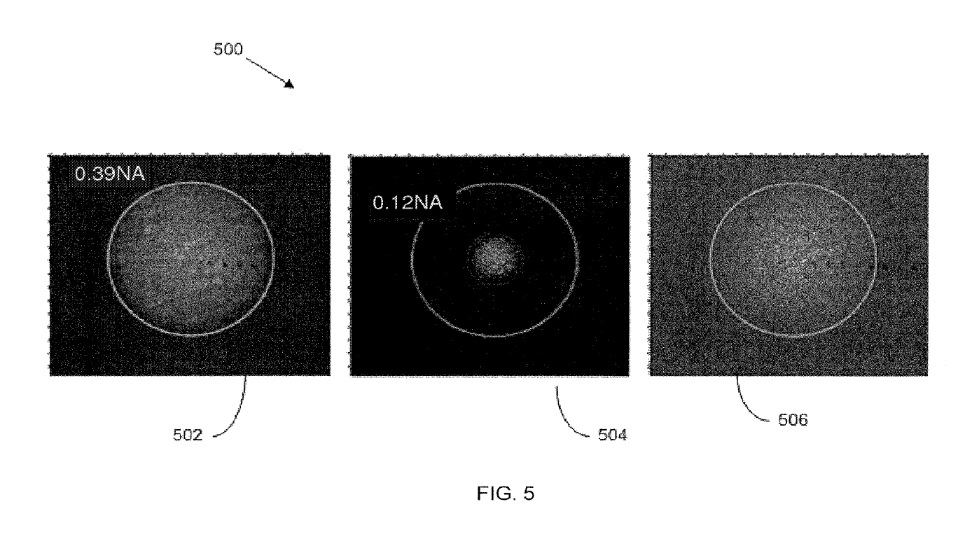

Turning now to FIG. 5, light distribution using known launching techniques can be seen in comparison to the optical output using a fiber mode scrambling device 400. Distribution chart 500 shows a measurement of a guide limited distribution using a standard lamp based light source launched into a 0.39 numerical aperture (NA), 400-micron core, step-index multimode fiber 502. It can be seen in distribution chart 500 that the fiber 502 is illuminated in near equilibrium conditions. Further, distribution chart 500 illustrates that the full spatial and angular extent of the fiber can be utilized. Distribution chart 504 illustrates the distribution when the same fiber 502 as that in distribution chart 50 is illuminated using a laser having a 0.12 numerical aperture. In this example, only a few modes of the fiber 502 are illuminated and the resulting spatial and angular profile can be smaller with little or no uniformity. Finally, distribution chart 506 illustrates the distribution when the fiber 502 is illuminated using the mode scrambling device shown in FIG. 4. In this example, transmission through the fiber 502 was measured to be about 98%.

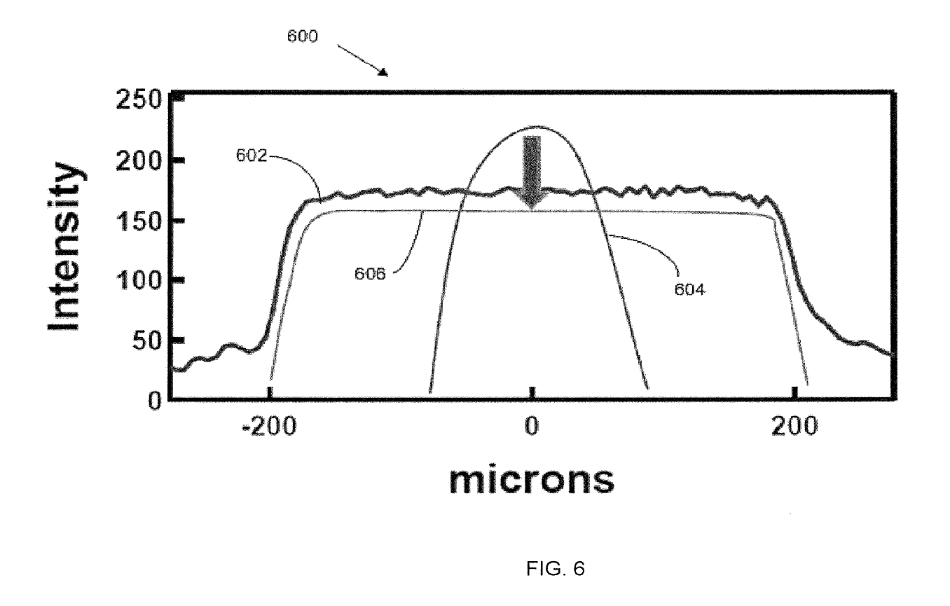

Turning to FIG. 6, an equilibrium intensity profile of step-index multimode optical fiber for the three modes of transmission in FIG. 5 can be seen. Illumination profile 600 shows the distribution of light through a 400 micron fiber core, as discussed above. Profile 602 illustrates light intensity through the fiber core when using an incandescent lamp launched into a 0.39 NA. It can be seen that the intensity is generally consistent across the entire 400 micron diameter of the fiber core. This general consistency can represent an ideal profile for a NIRS application. Profile 604 illustrates light intensity through the fiber core using a source having a limited spatial and/or angular transmission ability. For example, a laser as shown in FIG. 5. This type of source can produce a non-uniform profile which can be undesirable for NIRS applications. Profile 606 illustrates a converted non-ideal profile (e.g. profile 604). In one configuration, the converted non-ideal profile 606 can be generated using a scrambling device, such as that shown in FIG. 4.

In one embodiment, a scrambler can compress fiber optic cable and therefore the interface between the core of the fiber optic cable and the cladding within the fiber optic cable. This compression of the fiber optic cable can distort the cable to enable light from the modes illuminated by the source to leak into other propagating modes of the fiber optic cable. This can create nearly ideal coupling between the modes of the fiber optic cable such that the substantial majority of the light can enter the propagating modes resulting in an more equal distribution of modes within the fiber optic cable. Further, by utilizing more of the propagating modes of the fiber optic cable, the light can have a larger angular and spatial extent and uniformity within the fiber core, which can cause a larger angular and spatial extent when the light exists the fiber optic cable. Profile 600 illustrates the effect of using a scrambling device executing the above methodology to convert the Gaussian-like distribution of limited numerical aperture and spatial size (profile 604) into a flat-top profile 606. This converted non-ideal profile 606 can allow sources such as lasers and other limited spatial and/or angular light sources to provide the maximum total permissible optical exposure.

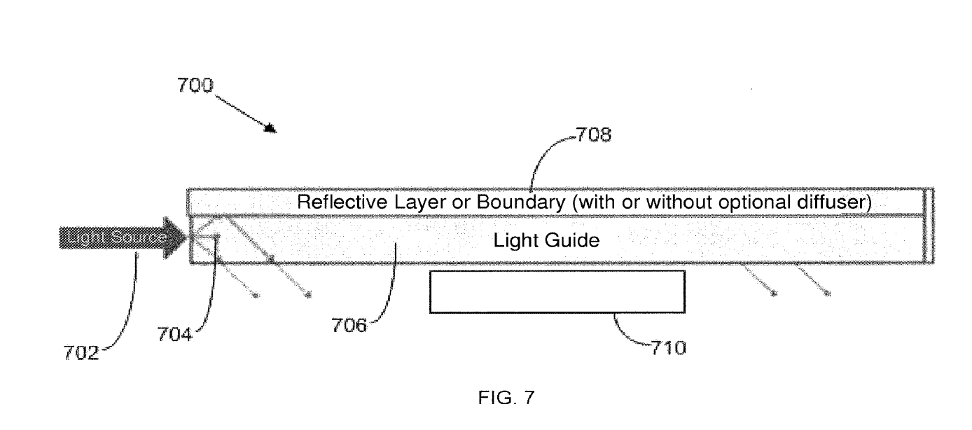

Direct lit systems, such as those described above, can rely on delivering light to a subject by either direct contact of the light source to the subject, or, via intermediate optics, such as prisms and/or lenses. While effective, these structures can be bulky in size, making them uncomfortable for use for some applications. Furthermore, larger probes can be more difficult to attach to a subject, are difficult to keep still, and can more easily detach from the subject. This can be of particular relevance when a probe is placed on a human head, or used with pediatric subjects and infants. Movement of the optical probe can have a deleterious effect on the operation of the device as motion can degrade or impact the measured signals. In order to reduce the size and bulk of direct lit systems, side lit configurations, such as those seen in FIGS. 7-18 can be used to keep the light source in a parallel direction to the subject, thereby reducing additional bulk associated with direct lit systems. This side lighting arrangement can reduce the size of an optical probe, and continue to maintain high permissible optical power density levels as discussed above. In one embodiment, side lit optical probes can be rigid. Alternatively, side lit optical probes can be flexible. Similar techniques can be used to collect light from a subject in the form of a receiver. The receivers can collect light from the patient and transport the light to light guides on a side of the device, which can then transmit the light to a detector. By reducing the size, the optical probes can be more stable and thereby result in greater signal fidelity. Furthermore, the reduced size can provide additional comfort for the subject and leave more space for other clinical devices as well as higher optical channel density within an optical probe. Smaller size optical probes can be more beneficial for use on infants and pediatric subjects.

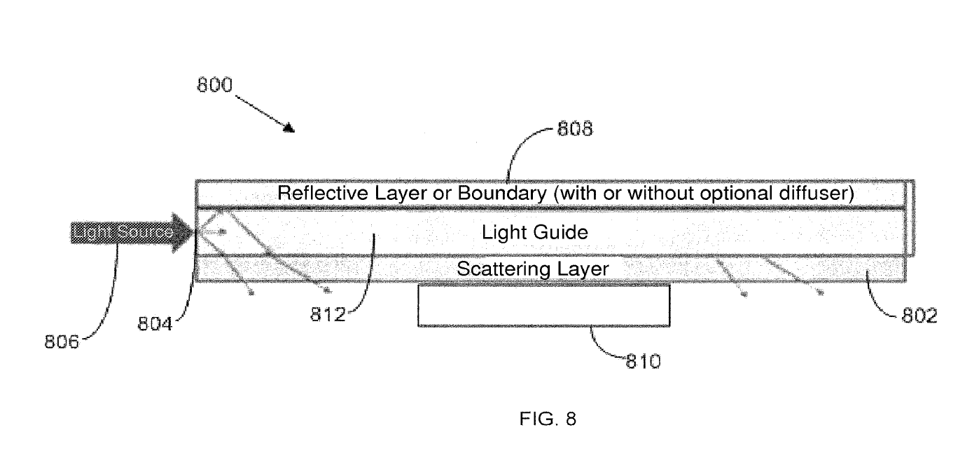

FIG. 7 illustrates a side-lit optical probe 700. A light source 702 projects a light 704 into the side of a light guide 706 contained within the optical probe 700. In one embodiment, a reflective layer 708 can be arranged on a top side of the optical probe 700. Alternatively the reflective layer 708 can be arranged on a top side of the optical probe 700 and extend around the lateral sides of the light guide 706. In one embodiment, the reflective layer 708 can extend around the lateral sides of the light guide 706, covering all portions of the light guide 706 not intended to be in contact with a subject 710. While the orientation and configuration of the reflexive layer 708 is discussed in regards to the optical probe of FIG. 7, the above orientations and configurations are applicable to all the side-lit optical probes described in FIGS. 8-18 as well. In one configuration, reflective layer 708 can include a diffuser element as discussed above. Alternatively, in some configurations, the reflective layer 708 can be used without a diffuser element. The light 704 can be reflected by the reflective layer 708 and directed to the subject 710. FIG. 8 illustrates a similar side lit optical probe 800 to that shown in FIG. 7; however, optical probe 800 can further include a scattering layer 802. Scattering layer 802 can scatter the light 804 received from the light source 806. Scattering layer 802 can further scatter the light 804 reflected from a reflective layer 808. Scattering the light with the scattering layer 802 can increase the angular divergence and distribution of the light 804 before it is transmitted towards a subject 810. In one configuration, the scattering layer 802 can be a Teflon sheet. Alternatively, the scattering layer 802 can be a filter or an attenuator. The scattering layer 802 can also be implemented by providing side-lighting from multiple directions, such as by using a lossy cladded or coreless optical fiber around the circumference of the light guide 812. In one example, the Teflon sheet can be 125 microns in thickness to about 250 microns in thickness. Alternatively, the Teflon sheet can be less than 125 microns or more than 250 microns in thickness. For example, the Teflon sheet may be between 100 microns and 300 microns thick.

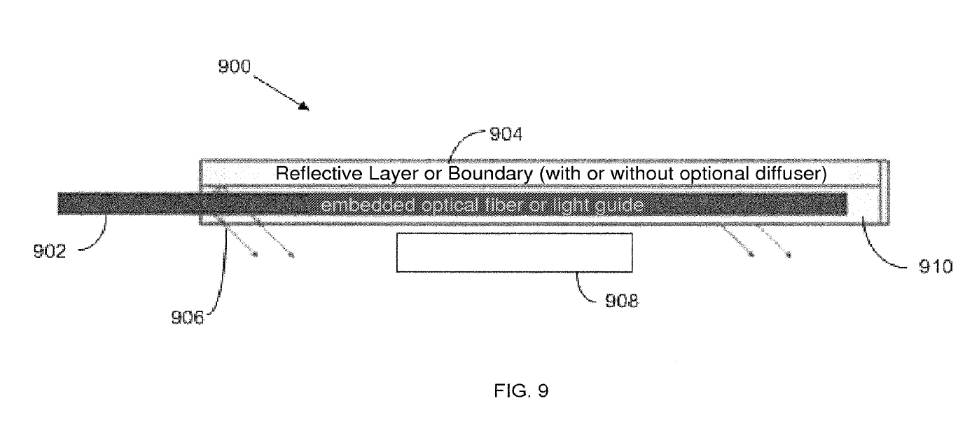

FIG. 9 shows a side-lit optical probe 900 having a light source 902 that is positioned parallel to a reflective layer 904 for transmitting light 906 though the optical probe 900. In one configuration, the light source 902 can be positioned to extend for the entire length of the reflective layer 904. Alternatively, the light guide 902 can extend for only a portion of the reflective layer 904. In one configuration, the light guide 902 can be a fiber optic cable. In one configuration, the reflective layer 904 can reflect the light 906 transmitted by the light guide 902 towards a subject 908. Furthermore, light 906 emitted from the light source 902 can be directly transmitted to a subject 908 as well. Additionally, the light source 902 can be disposed within a light guide 910.

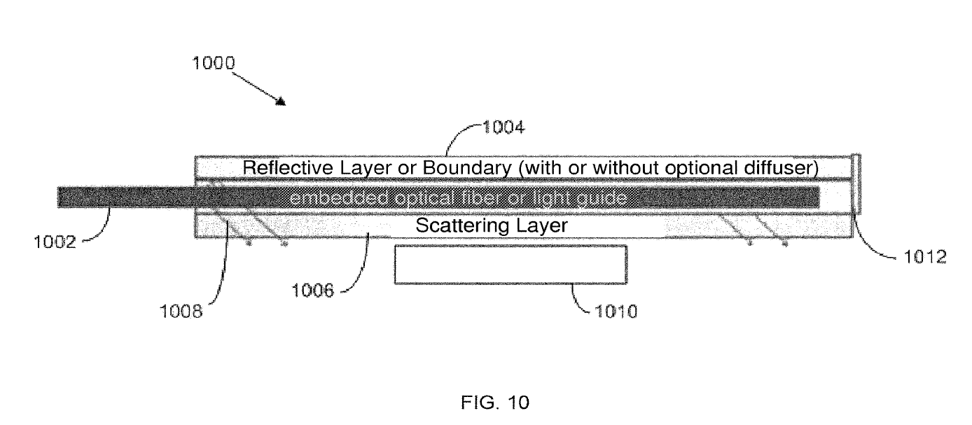

FIG. 10 shows a similar side lit optical probe 1000 having a light source 1002 that is positioned parallel to a reflective layer 1004. Optical probe 1000 can also include a scattering layer 1006. Scattering layer 1006 can scatter a light 1008 transmitted by the light source 1002. Scattering layer 1006 can further scatter the light 1008 reflected from the reflective layer 1004. Scattering the light with the scattering layer 1006 can increase the angular divergence and distribution of the light 1008 before it is transmitted towards a subject 1010. Additionally, the light source 1002 can be disposed within a light guide 1012. In one configuration, scattering layer 1006 can be a Teflon sheet. Alternatively, the scattering layer 1006 can be a filter or an attenuator. The scattering layer 1006 can also be implemented by providing side-lighting from multiple directions, such as by using a lossy cladded or coreless optical fiber around the circumference of the light source 1002. In one example, the Teflon sheet can be 125 microns in thickness to about 250 microns in thickness. Alternatively, the Teflon sheet can be less than 125 microns or more than 250 microns in thickness. For example, the Teflon sheet may be between 100 microns and 300 microns thick.

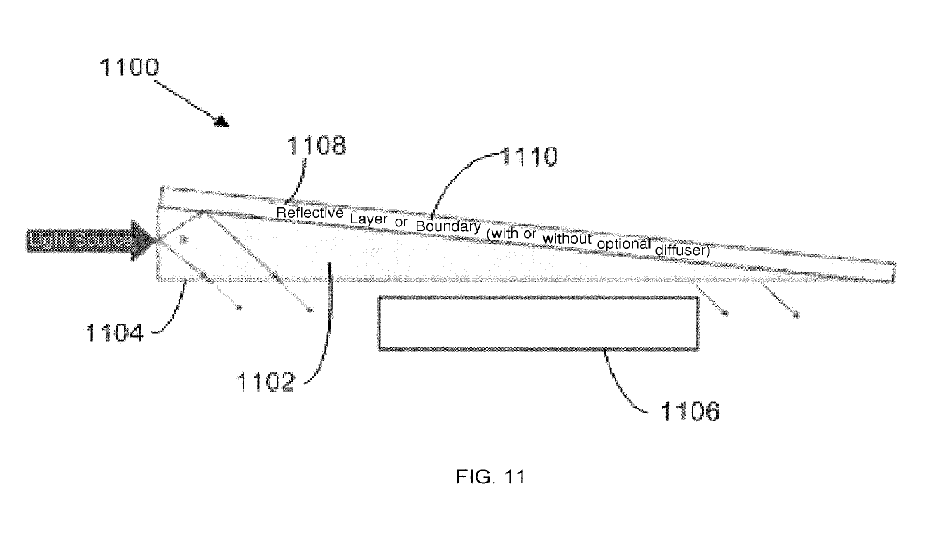

FIG. 11 illustrates an optical probe 1100 having an angular light guide 1102. A first surface 1104 can be adjacent to the subject 1106 and a second surface 1108 of the light chamber can be positioned at an acute angle to the first surface 1106 of the light chamber as shown in FIG. 11. In one configuration, the first surface 1104 and the second surface 1106 can be generally planar. However, it should be known that in some configurations, at least one of the first surface 1104 or the second surface 1106 may be non-planar. For example, the first surface 1104 can be constructed using a flexible material, and therefore, may deform when placed in contact with a subject 1108. For example, where the optical probe 1100 is placed against a portion of the subject 1106, such as a human head. In one configuration, a reflective layer 1110 can be positioned adjacent with and parallel to the second surface 1106. In one configuration, the reflective layer 1110 can include a diffuser element as discussed above. Alternatively, in some configurations, the reflective layer 1110 can be used without a diffuser element.

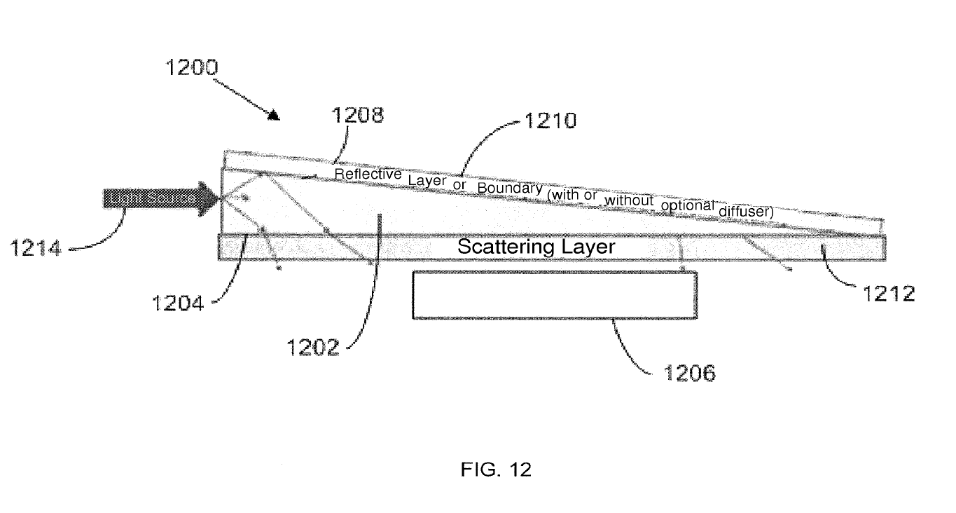

FIG. 12 illustrates an optical probe 1200 having an angular light guide 1202. A first surface 1204 can be adjacent to the subject 1206 and a second surface 1208 of the light chamber can be positioned at an acute angle to the first surface 1204 of the light guide as shown in FIG. 10. In one configuration, the first surface 1204 and the second surface 1208 can be generally planar. However, it should be known that in some configurations, at least one of the first surface 1204 or the second surface 1208 may be non-planar. For example, the first surface 1204 can be constructed using a flexible material, and therefore, may deform when placed in contact with a subject 1206. In one configuration, a reflective layer 1210 can be positioned adjacent to and parallel with the second surface 1208. In one configuration, the reflective layer 1210 can include a diffuser element as discussed above. Alternatively, in some configurations, the reflective layer 1210 can be used without a diffuser element. Optical probe 1200 can further include a scattering layer 1212. Scattering layer 1212 can scatter light received from a light source 1214. Scattering layer 1212 can further scatter light reflected from the reflective layer 1210. Scattering light with scattering layer 1212 can increase the angular divergence and distribution of the light before it is transmitted towards a subject 1206. In one configuration, the scattering layer 1212 can be a Teflon sheet. Alternatively, the scattering layer 1212 can be a filter or an attenuator. The scattering layer 1212 can also be implemented by providing side-lighting from multiple directions, such as by using a lossy cladded or coreless optical fiber around the circumference of light guide 1202. In one embodiment, the Teflon sheet can be 125 microns in thickness to about 250 microns in thickness. Alternatively, the Teflon sheet can be less than 125 microns or more than 250 microns in thickness. For example, the Teflon sheet may be between 100 microns and 300 microns thick.

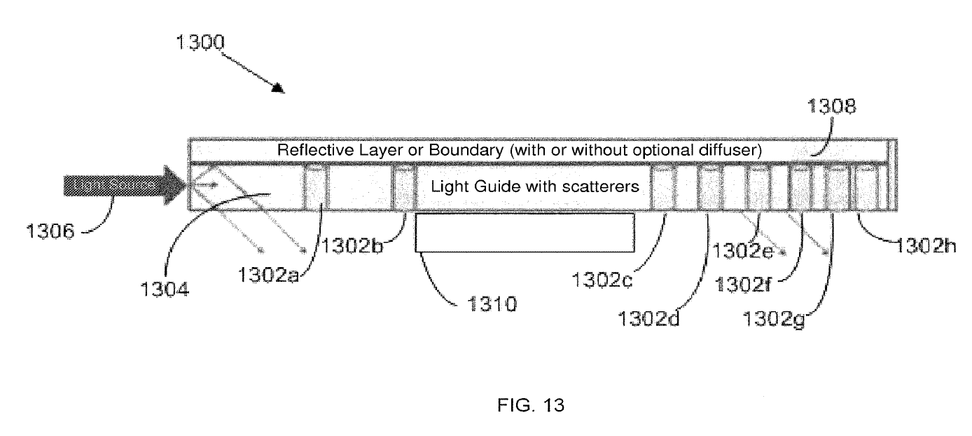

FIG. 13 illustrates a side-lit optical probe 1300 having a plurality of light scattering devices 1302a-h located within the light guide 1304. A light source 1306 projects a light into the side of the light guide 1304 contained within the optical probe 1300. As non-limiting examples, the light guide 1304 can be a gel, a polymer, or free space. On a top side of the optical probe 1300 can be a reflective layer 1308. In one configuration, reflective layer 1308 can include a diffuser element as discussed above. Alternatively, in some configurations, the reflective layer 1308 can be used without a diffuser element. The light projected by the light source 1306 can be reflected by the reflective layer 1308 and directed to a subject 1310. Additionally, the reflective layer 1308 can redirect light which may have been scattered away from the subject back to the subject. Furthermore, the plurality of light scattering devices 1302a-h can serve to further distribute the light received from the light source 1306. In one configuration, the plurality of scattering devices 1302a-h can be spaced at predetermined distances from each other along a linear plane generally parallel with a first surface 1312 of the optical probe 1300. In one example, the plurality of scattering devices 1302a-h can be spaced apart at equal distances. Alternatively, the plurality of scattering devices 1302a-h can be spaced apart at unequal distances. Scattering devices 1302a-h can distribute the light to provide a more uniform delivery of light to the subject 1310, leading to a greater amount of light over the surface area of the subject 1310. This uniform delivery (and subsequent collection) can aid in averaging out the effect of small superficial features on the subject 1310. For example, hair follicles, differences in pigmentation, blood vessels, etc, which can adversely impact the desired signal originating from deeper in the tissue of the subject. Furthermore, the scattering devices 1302a-h can be more efficient at changing the direction of light from the light source 1306, allowing for reduced size of the optical probe 1300.

The scattering devices 1302a-h can further aid in redirecting light traveling in a plane from the light source 1306 (i.e. shown as horizontal in FIG. 12), to a more non-planar direction (i.e. vertical, as shown in FIG. 12) in order to be delivered to a subject. The scattering devices 1302a-h can be discrete objects with differing indices of refraction or reflection. For example, structures fabricated by microlithography.

Alternatively, the scattering devices 1302a-h can be microscopic and dispersed in the material of the light guide 1304. Non-limiting examples can include microspheres or materials like titanium dioxide. The distribution, spacing, and/or concentration of the scattering devices 1302a-h can be positioned in a uniform or non-uniform pattern within the light guide 1304. Where the scattering devices 1302a-h are placed in a non-uniform pattern, the gradient of the scattering devices 1302a-h (i.e. the distance from light source 1306) can aid in the uniform distribution of light from the optical probe 1300. As the fluence of light is greatest nearest the light source 1306 and decreased rapidly with distance away from the light source 1306, fewer scattering devices 1302a-h are required near the light source 1306. More scattering devices 1302a-h can therefore be required further from the light source 1306 to deliver similar amounts of light to the subject across the surface of the light guide 1304.

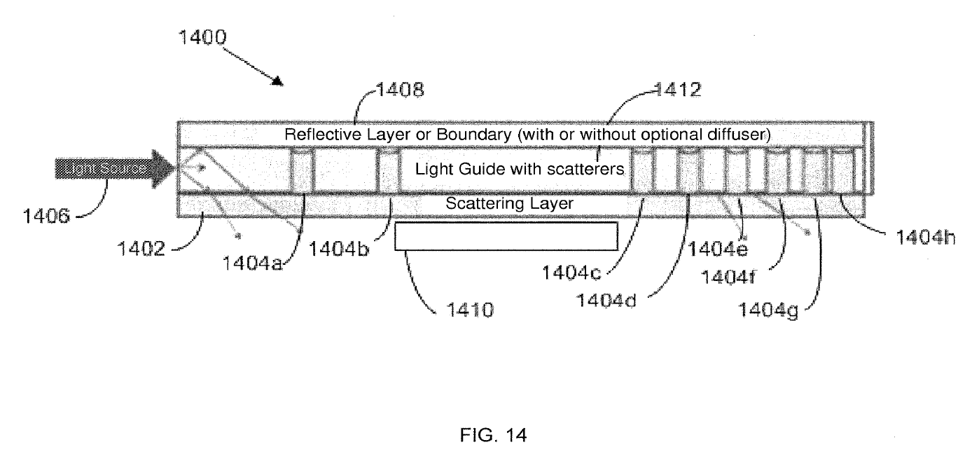

FIG. 14 illustrates a similar side-lit optical probe 1400 to that shown in FIG. 13; however, optical probe 1400 can further include a scattering layer 1402 in addition to the plurality of scattering devices 1404a-h. Scattering layer 1402 can further scatter the light received from a light source 1406. Scattering layer 1402 can also further scatter the light reflected from reflective layer 1408. Scattering the light with the scattering layer 1402 can increase the angular divergence and distribution of the light before it is transmitted towards a subject 1410. Additionally, the scattering layer 1402 in combination with the plurality of scattering devices 1404a-h can more effectively scatter the light than using a scattering layer 1402 only. In one configuration, the scattering layer 1402 can be a Teflon sheet. The Teflon sheet can be 125 microns in thickness to about 250 microns in thickness. Alternatively, the Teflon sheet can be less than 125 microns or more than 250 microns in thickness. For example, the Teflon sheet may be between 200 microns and 300 microns thick. The scattering devices 1404a-h can be discrete objects with differing indices of refraction or reflection. For example, structures fabricated by microlithography. Alternatively, the scattering devices 1404a-h can be microscopic and dispersed in the material of a light guide 1410. Non-limiting examples can include microspheres or materials like titanium dioxide. The distribution, spacing, and/or concentration of the scattering devices 1404a-h can be positioned in a uniform or non-uniform pattern within the light guide 1412.

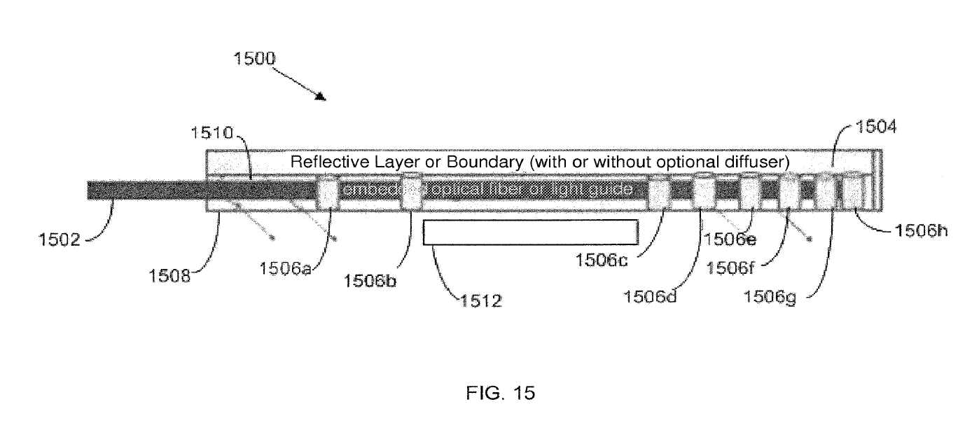

FIG. 15 shows a side-lit optical probe 1500 having a light source 1502 that is positioned parallel to a reflective layer 1504 for transmitting light though the optical probe 1500. In one configuration, the light source 1502 can be positioned to extend for the entire length of the reflective layer 1504. Alternatively, the light source 1502 can extend for only a portion of the reflective layer 1504. In one configuration, the light source 1502 can be a fiber optic cable. The optical probe 1500 can further include a plurality of scattering devices 1506a-h. The scattering devices 1506a-h can be discrete objects with differing indices of refraction or reflection. For example, structures fabricated by microlithography. Alternatively, the scattering devices 1506a-h can be microscopic and dispersed in the material of a light guide 1510. Non-limiting examples can include microspheres or materials like titanium dioxide. The distribution, spacing, and/or concentration of the scattering devices 1506a-h can be positioned in a uniform or non-uniform pattern within the light guide 1510. The plurality of light scattering devices 1506a-h can serve to further distribute the light received from the light source 1502. In one configuration, the plurality of scattering devices 1506a-h can be spaced at predetermined distances from each other along a linear plane generally parallel with a first surface 1508 of the optical probe 1500. In one example, the plurality of scattering devices 1506a-h can be spaced apart at equal distances. Alternatively, the plurality of scattering devices 1506a-h can be spaced apart at unequal distances. In one configuration, the plurality of scattering devices 1506a-h can be positioned on only one side of the light source 1502. Alternatively, the plurality of scattering devices 1506a-h can be positioned on both sides of the light source 1502. Where the plurality of scattering devices 1506a-h are positioned on either side of the light guide 1502, the light received from the light source 1502 can be more effectively scattered than where the scattering devices are located on only one side of the light guide 1502. In one embodiment, the reflective layer 1504 can reflect the light 1506a-h transmitted by the light guide 1502 towards a subject 1512. Furthermore, light emitted from the light source 1502 can be directly transmitted to a subject 1512 as well. Additionally, the light source 1502 can be disposed within the light guide 1510.

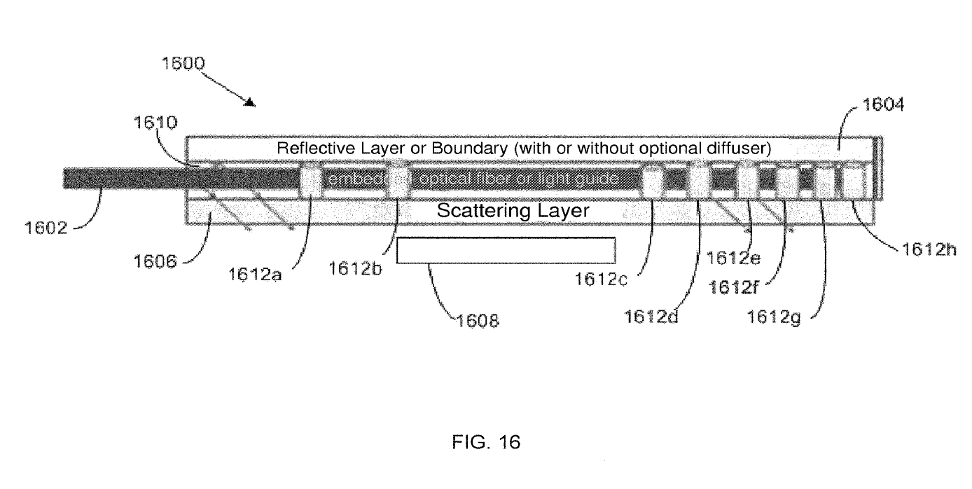

FIG. 16 shows a similar side-lit optical probe 1600 having a light source 1602 that is positioned parallel to a reflective layer 1604. Optical probe 1600 can also include a scattering layer 1606. Scattering layer 1606 can scatter a light transmitted by the light guide 1602. Scattering layer 1606 can further scatter the light reflected from the reflective layer 1604. Scattering the light with the scattering layer 1606 can increase the angular divergence and distribution of the light before it is transmitted towards a subject 1608. Additionally, the light source 1602 can be disposed within a light guide 1610.

Additionally, optical probe 1600 can further include a plurality of scattering devices 1612a-h. The scattering devices 1610a-h can be discrete objects with differing indices of refraction or reflection. For example, structures fabricated by microlithography. Alternatively, the scattering devices 1610a-h can be microscopic and dispersed in the material of a light guide 1610. Non-limiting examples can include microspheres or materials like titanium dioxide. The distribution, spacing, and/or concentration of the scattering devices 1612a-h can be positioned in a uniform or non-uniform pattern within the light guide 1610. The plurality of light scattering devices 1612a-h can serve to further distribute the light received from the light source 1602. The scattering layer 1606 in combination with the plurality of scattering devices 1612a-h can more effectively scatter the light than using a scattering layer 1606 only. In one configuration, the scattering layer 1606 can be a Teflon sheet. The Teflon sheet can be about 125 microns in thickness to about 250 microns in thickness. Alternatively, the Teflon sheet can be less than 125 microns or more than 250 microns in thickness. For example, the Teflon sheet may be between 100 microns and 300 microns thick.

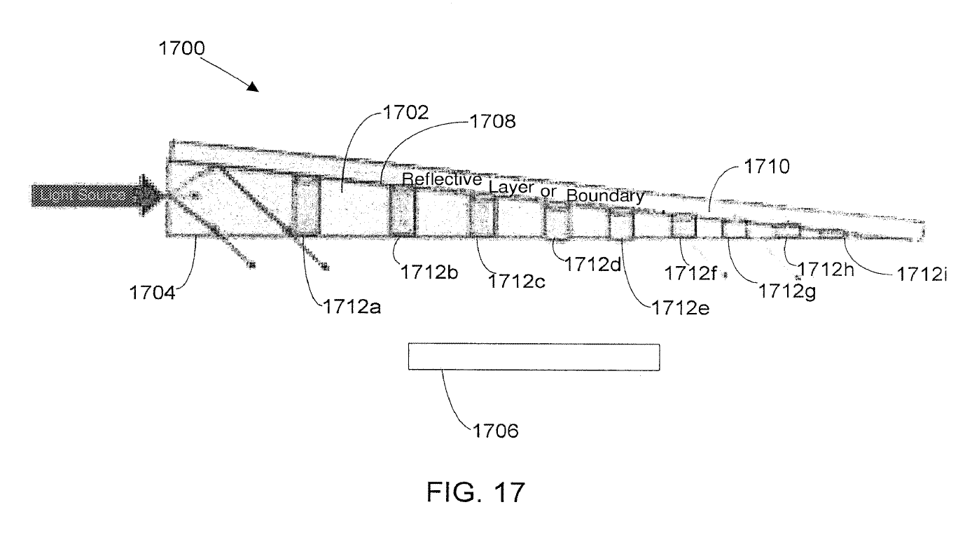

FIG. 17 illustrates an optical probe 1700 having an angular light guide 1702. A first surface 1704 can be adjacent to a subject 1706 and a second surface 1708 of the light guide 1702 can be positioned at an acute angle to the first surface 1706 of the light guide 1702 as shown in FIG. 17. In one configuration, the first surface 1704 and the second surface 1706 can be generally planar. However, it should be known that in some embodiments, at least one of the first surface 1704 or the second surface 1706 may be non-planar. For example, the first surface 1704 can be constructed using a flexible material, and therefore, may deform when placed in contact with the subject 1706. For example, when the optical probe is placed on a portion of the subject 1706, such as a human head. In one configuration, a reflective layer 1710 can be positioned adjacent with and parallel to the second surface 1706. In one configuration, the reflective layer 1710 can include a diffuser element as discussed above. Alternatively, in some configurations, the reflective layer 1710 can be used without a diffuser element.

Optical probe 1700 can further include a plurality of scattering devices 1712a-h located within the light chamber 1702. The plurality of light scattering devices 1712a-i can serve to further distribute the light received from a light source 1714. The scattering devices 1712a-i can be discrete objects with differing indices of refraction or reflection. For example, structures fabricated by microlithography. Alternatively, the scattering devices 1712a-i can be microscopic and dispersed in the material of a light guide 1702. Non-limiting examples can include microspheres or materials like titanium dioxide. The distribution, spacing, and/or concentration of the scattering devices 1712a-i can be positioned in a uniform or non-uniform pattern within the light guide 1702. The plurality of light scattering devices 1712a-i can serve to further distribute the light received from the light guide 1702. In one configuration, the plurality of scattering devices 1712a-i can be spaced at predetermined distances from each other along a linear plane generally parallel with the first surface 1704 of the optical probe 1700. In one example, the plurality of scattering devices 1712a-i can be spaced apart at equal distances. Alternatively, the plurality of scattering devices 1712a-i can be spaced apart at unequal distances. In one configuration, the plurality of scattering devices 1712a-i can extend along an axis perpendicular to the first surface 1704 and extend towards the second surface 1708. In one configuration, the plurality of scattering devices 1712a-i can extend from the first surface 1704 to the second surface 1708. However, in other configurations, the plurality of scattering devices 1712a-i may only extend through part of the distance between the first surface 1704 and the second surface 1708.

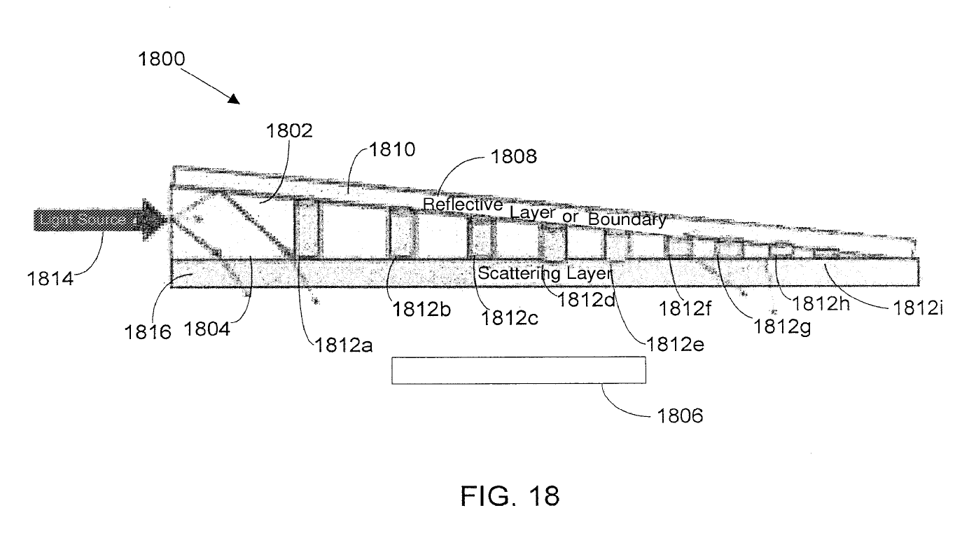

FIG. 18 illustrates an optical probe 1800 having an angular light guide 1802. A first surface 1804 can be adjacent to a subject 1806 and a second surface 1808 of the light guide 1802 can be positioned at an acute angle to the first surface 1804 of the light chamber as shown in FIG. 17. In one configuration, the first surface 1804 and the second surface 1808 can be generally planar. However, it should be known that in some configurations, at least one of the first surface 1804 or the second surface 1808 may be non-planar. For example, the first surface 1804 can be constructed using a flexible material, and therefore, may deform when placed in contact with the subject 1806. In one configuration, a reflective layer 1810 can be positioned adjacent with and parallel to the second surface 1806. In one configuration, the reflective layer 1810 can include a diffuser element as discussed above. Alternatively, in some configurations, the reflective layer 1810 can be used without a diffuser element.

Optical probe 1800 can further include a plurality of scattering devices 1812a-i located within the light guide 1802. The plurality of light scattering devices 1812a-i can serve to further distribute the light received from a light source 1814. The scattering devices 1812a-i can be discrete objects with differing indices of refraction. For example, structures fabricated by microlithography. Alternatively, the scattering devices 1812a-i can be microscopic and dispersed in the material of a light guide 1802. Non-limiting examples can include microspheres or materials like titanium dioxide. The distribution, spacing, and/or concentration of the scattering devices 1812a-i can be positioned in a uniform or non-uniform pattern within the light guide 1802. The plurality of light scattering devices 1812a-i can serve to further distribute the light received from the light guide 1802. In one configuration, the plurality of scattering devices 1812a-i can be spaced at predetermined distances from each other along a linear plane generally parallel with the first surface 1804 of the optical probe 1800. In one example, the plurality of scattering devices 1812a-i can be spaced apart at equal distances. Alternatively, the plurality of scattering devices 1812 a-i can be spaced apart at unequal distances. In one configuration, the plurality of scattering devices 1812a-i can extend along an axis perpendicular to the first surface 1804 and extend towards the second surface 1808. In one configuration, the plurality of scattering devices 1812 can extend from the first surface 1804 to the second surface 1808. However, in other configurations, the plurality of scattering devices 1812a-h may only extend through part of the distance between the first surface 1804 and the second surface 1808.

Optical probe 1800 can further include a scattering layer 1816. Scattering layer 1816 can scatter light received from the light source 1814. Scattering layer 1816 can further scatter light reflected from the reflective layer 1816. Scattering light with the scattering layer 1816 can increase the angular divergence and distribution of the light before it is transmitted towards a subject 1806. In one configuration, the scattering layer 1816 can be a Teflon sheet. The Teflon sheet can be 125 microns in thickness to about 250 microns in thickness. Alternatively, the Teflon sheet can be less than 125 microns or more than 250 microns in thickness. For example, the Teflon sheet may be between 100 microns and 300 microns thick. Furthermore, in one configuration the scattering layer 1816 in combination with the plurality of scattering devices 1812a-i can more effectively scatter the light than using a scattering layer 1816 only.

While the side-lit optical probes in FIGS. 7-18 are shown as transmitting light, it should be known that the optical probes in FIGS. 7-18 can be used for light delivery and/or light collection.

Additionally, while reference is made in this application to applying NIRS to human subjects, it should be known that NIRS techniques can also be applied to any biological entity, such as mammals, birds, reptiles, and the like.

The present invention has been described in terms of one or more preferred embodiments, and it should be appreciated that many equivalents, alternatives, variations, and modifications, aside from those expressly stated, are possible and within the scope of the invention.

1. An optical device, the optical device comprising:
a light source providing a light that is directed along a first axis;
a diffusive element positioned proximate to the light source to receive the light and to diffuse the light as it exits the diffusive element; and
a directional optical element directing the light exiting the diffusive element along at least one of the first axis and a second axis generally perpendicular to the first axis, to project the light out of the optical probe and onto a subject.

2. The optical device of claim 1, wherein the light source is a laser.

3. The optical device of claim 1, wherein the diffusive element is one of a surface diffusive element, a diffractive diffusive element, a refractive diffusive element, a holographic diffusive element and a phase diffusive element.

4. The optical device of claim 1, wherein the optical probe is a spectroscopy device.

5. The optical device of claim 4, wherein the optical probe is a near-infrared spectroscopy device.

6. The optical device of claim 1, wherein the light source uses a plurality of fiber optic cables to transmit light.

7. The optical device of claim 1, wherein the directional optical element is a prism.

8. A method of increasing light throughput in an optical probe, the method comprising:
transmitting a light along a first axis from a light source;
receiving the light through a diffusive element, the diffusive element positioned proximate to the light source to diffuse the light as it exits the diffusive element; and
directing the light using a directional element, the directional element directing the light exiting the diffusive element along at least one of the first axis and a second axis generally perpendicular to the first axis, to project the light out of the optical probe and onto a subject.

9. The method of claim 8, wherein the directional element is a prism.

10. The method of claim 8, wherein the direction of the light is altered by 90 degrees.

11. The method of claim 8, wherein the diffusive element is a Teflon sheet.

12. The method of claim 8, wherein the optical probe is a near-infrared spectroscopy device.

13. A side lit optical spectroscopy device, the device comprising:
a light source providing a light that is directed along a first axis into a light guide;
a reflective element, the reflective element positioned proximately along a first side of the light guide and configured to reflect the light from the light source towards a second side of the light guide; and
a scattering layer, the scattering layer positioned proximate to the second side of the light guide and configured to scatter the light from the light source and the light reflected by the reflective element prior to the light exiting the side lit optical spectroscopy device.

14. The device of claim 13, wherein a diffusive layer is disposed between the reflective layer and the first side of the light guide.

15. The device of claim 13, wherein the light source is a fiber optic cable.

16. The device of claim 13, further comprising a plurality of scattering devices, the plurality of scattering devices disposed within the light guide.

17. The device of claim 16, wherein the plurality of scattering devices are discrete objects with differing indices of refraction or reflection.

18. The device of claim 16, wherein the plurality of scatting devices are microspheres.

19. The device of claim 16, wherein the plurality of scattering devices are spaced at equal distances along the second side of the light guide.

20. The device of claim 13, wherein the first side of the light guide is parallel to the second side of the light guide.
